(19) United States
(12) Patent Application Publication
(10) Pub. No.: US 2022/0260820 A1
(43) Pub. Date: Aug. 18, 2022

(54) COMBINED REFLECTANCE CONFOCAL AND TWO-PHOTON MICROSCOPY SYSTEM FOR HIGH-SPEED HIGH-CONTRAST CELLULAR EXAMINATION OF LIVING TISSUE AND METHOD FOR HIGH-SPEED/HIGH-CONTRAST CELLULAR EXAMINATION OF LIVING TISSUE USING THE SAME

(71) Applicants: POSTECH Research and Business Development Foundation, Pohang-si (KR); University Industry Foundation, Yonsei University, Seoul (KR)

(72) Inventors: Ki Hean KIM, Pohang-si (KR); Byung Ho OH, Seoul (KR); Won Yeong PARK, Busan (KR); Bum Ju KIM, Pohang-si (KR); Soo Hyun PARK, Pohang-si (KR); Viet Hoan Le, Goyang-si (KR)

(73) Assignees: POSTECH Research and Business Development Foundation, Pohang-si (KR); University Industry Foundation, Yonsei University, Seoul (KR)

(21) Appl. No.: 17/672,730

(22) Filed: Feb. 16, 2022

Related U.S. Application Data
(60) Provisional application No. 63/150,021, filed on Feb. 16, 2021.

(51) Int. Cl. G02B 21/00; G01N 1/30; G02B 27/14; G01N 21/64; A61B 5/00

(52) U.S. Cl. CPC G02B 21/008; A61B 5/441; G02B 21/0076; G02B 21/0028; G02B 21/0072; G02B 21/0032; G02B 27/141; G01N 21/6458; G01N 21/6402; G01N 21/6428; A61B 5/0071; G01N 2001/302; G02B 21/0012; G01N 2021/6439; G01N 1/30

(57) ABSTRACT

A combined reflectance confocal and two-photon microscopy system for non-invasive high-speed/high-contrast examination of living tissue and a method for non-invasive high-speed/high-contrast examination of living tissue using the same, wherein the combined reflectance confocal and two-photon microscopy system enables high speed imaging while providing extracellular matrix/cell contrast together with information of existing reflectance confocal microscopy.

### FIELD

The present invention relates to a combined reflectance confocal and two-photon microscopy system for non-invasive high-speed/high-contrast examination of living tissue and a method for non-invasive high-speed/high-contrast examination of living tissue using the same. More particularly, the present invention relates to a combined reflectance confocal and two-photon microscopy system for non-invasive high-speed/high-contrast examination of living tissue, which enables high speed imaging while providing extracellular matrix/cell contrast together with information of existing reflectance confocal microscopy, and a method for non-invasive high-speed/high-contrast examination of living tissue using the same.

### BACKGROUND

Reflectance confocal microscopy is a high-resolution cell imaging technique based on light scattering and is used as a cell examination method in a clinical environment by enabling non-invasive imaging.

In clinical fields, reflectance confocal microscopy is applied to examination of anterior segment in ophthalmology and detection of skin cancer in dermatology. In examination of the anterior segment, reflectance confocal microscopy is used for early diagnosis of a causative agent of keratitis through non-invasive imaging.

Since keratitis is caused by infection with various pathogens, such as bacteria, fungi, viruses, or protozoa and treatment varies depending on the causative agent, it is important to detect the infectious pathogens precisely for proper treatment.

The reflectance confocal microscopy enables detection of infection caused by amoeba and fungi having relatively large sizes and characteristic morphologies.

In dermatology, the boundary of non-melanoma skin cancer is non-invasively detected and used to guide skin cancer surgery. Detection of the boundary of skin cancer is important in precision surgery that requires minimal scarring, such as on the human face.

Since a tissue environment in skin cancer provides morphological change, such as cell clustering, as compared to a normal environment, reflectance confocal microscopy enables detection of skin cancer boundaries by monitoring scattering due to the morphological change.

However, such reflectance confocal microscopy provides only low-contrast information due to non-specific light scattering signals in living tissue. In particular, reflectance confocal microscopy provides low cell contrast in lesions where a micro-tissue structure is destroyed.

To overcome this problem, fluorescence confocal microscopy and auto-fluorescence-based two-photon microscopy were developed. However, fluorescence confocal microscopy requires exogenous fluorescence labeling and thus cannot be directly applied to the human body, and two-photon microscopy provides too weak auto-fluorescence signals to photograph a sufficiently large area during a given examination time

In order to overcome the limitation of reflectance confocal microscopy, multi-modal microscopy combined with fluorescence confocal microscopy to provide additional fluorescence information in tissue is being developed in the art.

However, auto-fluorescence-based microscopy makes it difficult to achieve high-speed imaging simultaneously with reflectance confocal microscopy due to weak auto-fluorescence signals and has limitations in clinical use due to toxicity even when cell fluorescent materials are used to improve imaging speed.

### RELATED LITERATURE

### Patent Document

(Patent Document 1) Korean Patent Publication No. 10-1898220 (Title of the Invention: Confocal microscope and image processing method using the same, Issue Date: Sep. 12, 2018)

### SUMMARY

Embodiments of the present invention are conceived to solve such problems in the art and it is an aspect of the present invention to provide a combined reflectance confocal and two-photon microscopy system for non-invasive high-speed/high-contrast examination of living tissue, which enables high speed imaging while providing both cell and extracellular matrix contrasts via clinically compatible cell labeling and intrinsic signals together with information of existing reflectance confocal microscopy, and a method for non-invasive high-speed/high-contrast examination of living tissue using the same.

It will be understood that aspects of the present invention are not limited to the above. The above and other aspects of the present invention will become apparent to those skilled in the art from the detailed description of the following embodiments in conjunction with the accompanying drawings.

In accordance with one aspect of the present invention, there is provided a combined reflectance confocal and two-photon microscopy system for non-invasive high-speed/high-contrast examination of living tissue, comprising: a light source emitting laser beams; an objective lens delivering light received from the light source to living tissue; a two-photon microscopy unit photographing cells and an extracellular matrix of the living tissue with effect light generated from the living tissue; a reflectance confocal microscopy unit photographing an interior of the living tissue with effect light reflected from the living tissue; an optical path guide guiding a traveling path of the laser beams from the light source to the objective lens and guiding the effect light generated and reflected from the living tissue to the two-photon microscopy unit and the reflectance confocal microscopy unit; and an image generator generating an image of the living tissue photographed by the two-photon microscopy unit and the reflectance confocal microscopy unit.

The optical path guide may include: a first optical filter allowing only a laser beam having a wavelength of 680 nm or more among the laser beams emitted from the light source to pass therethrough; a first lens group expanding the laser beams; a beam splitter allowing the laser beams traveling from the light source to the objective lens to pass therethrough and guiding the effect light to the reflectance confocal microscopy unit; a second lens group expanding the laser beams; and a first dichroic mirror allowing the laser beams traveling from the light source to the objective lens to pass therethrough and guiding the effect light to the two-photon microscopy unit and the reflectance confocal microscopy unit.

In accordance with another aspect of the present invention, there is provided a method for non-invasive high-speed/high-contrast examination of living tissue using a combined reflectance confocal and two-photon microscopy system for non-invasive high-speed/high-contrast examination of living tissue, the method including: a living tissue staining step in which living tissue is stained with moxifloxacin; an irradiation step in which a laser beam is emitted towards the living tissue; an effect light guide step in which effect light reflected from the living tissue and subjected to fluorescence excitation through moxifloxacin is guided to a reflectance confocal microscopy unit and a two-photon microscopy unit; an image generation step in which an image of the living tissue photographed by the two-photon microscopy unit and the reflectance confocal microscopy unit is generated, wherein the effect light guide step includes: a first effect light guide step in which, among the effect light reflected from the living tissue and subjected to fluorescence excitation through moxifloxacin, first effect light having a wavelength of less than 700 nm is reflected from a first dichroic mirror and guided to the two-photon microscopy unit among the effect light reflected from the living tissue and subjected to fluorescence excitation through moxifloxacin; and a second effect light guide step in which, among the effect light reflected from the living tissue, second effect light having a wavelength of 700 nm or more is allowed to pass through the first dichroic mirror and is guided to the reflectance confocal microscopy unit.

The irradiation step may include: a first light filtering step in which only a laser beam having a wavelength of 680 nm or more among the laser beams emitted from the light source is allowed to pass through a first optical filter; a first beam expansion step in which the laser beam is primarily expanded by a first lens group; a first beam passing step in which the laser beam subjected to primary expansion is allowed to pass through a beam splitter; a second beam expansion step in which the laser beam is secondarily expanded by a second lens group; a second beam passing step in which the laser beam subjected to secondary expansion is allowed to pass through a first dichroic mirror; and a living tissue irradiation step in which the laser beam having passed through the first dichroic mirror is delivered to the living tissue through an objective lens.

The combined reflectance confocal and two-photon microscopy system and the method for non-invasive high-speed/high-contrast examination of living tissue using the same according to the present invention provide the following effects.

First, the microscopy system and the method according to the present invention can provide extracellular matrix/cell contrast through staining of living tissue with moxifloxacin, reflection, fluorescence excitation, and multi-imaging of the living tissue having second harmonic contrast.

Secondly, the microscopy system and the method according to the present invention enable accurate imaging of a cell structure by providing information on cell membranes (reflected signal) and cytoplasm (fluorescence signal) of epidermal cells at the same time upon simultaneous in-vivo photographing of living tissue stained with moxifloxacin using a two-photon microscopy unit and a reflectance confocal microscopy unit.

Thirdly, the microscopy system and the method according to the present invention enable detection of a skin microenvironment with high contrast by allowing the structure of cell membranes and an extracellular matrix to be photographed based on reflected signals in a relatively deep dermal layer while providing additional information on distribution of collagen in the cells and the matrix through a fluorescence/second harmonic signal.

Fourthly, the microscopy system and the method according to the present invention are advantageously used for detection and diagnosis of a causative agent of a disease through additional comparison between the cells and the extracellular matrix.

Fifthly, the microscopy system and the method according to the present invention can be advantageously used not only in various clinical fields including ophthalmology and dermatology, but also in generation of images for accurate detection and diagnosis through direct imaging of a surgical site during surgery.

Sixthly, the microscopy system and the method according to the present invention enable diagnosis through moxifloxacin-based fluorescence/second harmonic images using a two-photon microscopy unit in a clinical environment where it is difficult to perform diagnosis based only on reflected signals, as in a diseased cornea.

Seventhly, the microscopy system and the method according to the present invention can provide multi-contrast images at high speed using a resonant scanner and a high-speed photomultiplier tube.

It will be understood that advantageous effects of the present invention are not limited to the above effects, and the above and other advantageous effects of the present invention will become apparent to those skilled in the art from the detailed description of the following embodiments in conjunction with the accompanying drawings.

### DRAWINGS

The above and other aspects, features, and advantages of the present invention will become apparent from the detailed description of the following embodiments in conjunction with the accompanying drawings:

FIG. 1 is a block diagram of a combined reflectance confocal and two-photon microscopy system for non-invasive high-speed/high-contrast examination of living tissue according to the present invention.

FIG. 2 is a schematic view of an optical path guide of the combined reflectance confocal and two-photon microscopy system for non-invasive high-speed/high-contrast examination of living tissue according to the present invention.

FIG. 3 is a schematic view of an optical path guided by a first dichroic mirror in the optical path guide of the combined reflectance confocal and two-photon microscopy system for non-invasive high-speed/high-contrast examination of living tissue according to the present invention.

FIG. 4 is a schematic view of a two-photon microscopy unit of the combined reflectance confocal and two-photon microscopy system for non-invasive high-speed/high-contrast examination of living tissue according to the present invention.

FIG. 5 is a schematic view of a reflectance confocal microscopy unit of the combined reflectance confocal and two-photon microscopy system for non-invasive high-speed/high-contrast examination of living tissue according to the present invention.

FIG. 6 is a flowchart of a non-invasive high-speed/high-contrast examination method using the combined reflectance confocal and two-photon microscopy system for non-invasive high-speed/high-contrast examination of living tissue according to the present invention.

FIG. 7 is a flowchart of an irradiation step in the non-invasive high-speed/high-contrast examination method using the combined reflectance confocal and two-photon microscopy system for non-invasive high-speed/high-contrast examination of living tissue according to the present invention.

FIG. 8 is a flowchart of a first effect light guide step in the non-invasive high-speed/high-contrast examination method using the combined reflectance confocal and two-photon microscopy system for non-invasive high-speed/high-contrast examination of living tissue according to the present invention.

FIG. 9 is a flowchart of a second effect light guide step in the non-invasive high-speed/high-contrast examination method using the combined reflectance confocal and two-photon microscopy system for non-invasive high-speed/high-contrast examination of living tissue according to the present invention.

FIG. 10 is two-photon and reflection confocal images of the skin of a mouse ear stained with moxifloxacin, as obtained through photographing by the combined reflectance confocal and two-photon microscopy system for non-invasive high-speed/high-contrast examination of living tissue according to the present invention.

FIG. 11 is two-photon and reflection confocal images of the cornea of a mouse stained with moxifloxacin, as obtained through photographing by the combined reflectance confocal and two-photon microscopy system for non-invasive high-speed/high-contrast examination of living tissue according to the present invention.

FIG. 12 is two-photon and reflection confocal images of the cornea and the skin of a mouse stained with moxifloxacin, as obtained through photographing by the combined reflectance confocal and two-photon microscopy system for non-invasive high-speed/high-contrast examination of living tissue according to the present invention.

FIG. 13 is two-photon and reflection confocal images of human basal cell carcinoma (basal cell carcinoma) stained with moxifloxacin and a combined image thereof, as obtained through photographing by the combined reflectance confocal and two-photon microscopy system for non-invasive high-speed/high-contrast examination of living tissue according to the present invention.

FIG. 14 shows (A) which is a view illustrating a mechanism of two-photon excitation fluorescence in the combined reflectance confocal and two-photon microscopy system for non-invasive high-speed/high-contrast examination of living tissue according to the present invention and the method for non-invasive high-speed/high-contrast examination of living tissue using the same, and (B) which is a view illustrating a mechanism of generating second harmonic signals in the combined reflectance confocal and two-photon microscopy system for non-invasive high-speed/high-contrast examination of living tissue according to the present invention and the method for non-invasive high-speed/high-contrast examination of living tissue using the same.

FIG. 15 shows (A) which is is a view of a two-photon florescence-based excitation spectrum in the combined reflectance confocal and two-photon microscopy system for non-invasive high-speed/high-contrast examination of living tissue according to the present invention and the method for non-invasive high-speed/high-contrast examination of living tissue using the same, and (B) which is a view of a fluorescence emission spectrum in the combined reflectance confocal and two-photon microscopy system for non-invasive high-speed/high-contrast examination of living tissue according to the present invention and the method for non-invasive high-speed/high-contrast examination of living tissue using the same.

### DETAILED DESCRIPTION

Hereinafter, exemplary embodiments of the present invention will be described in detail with reference to the accompanying drawings. It should be understood that the present invention is not limited to the following embodiments and may be embodied in different ways, and that the embodiments are provided for complete disclosure and thorough understanding of the present invention by those skilled in the art. The scope of the present invention is defined only by the claims. Like components will be denoted by like reference numerals throughout the specification.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

When an element or layer is referred to as being “on,” “connected to,” or “coupled to” another element or layer, it may be directly on, connected to, or coupled to the other element or layer or intervening elements or layers may be present. However, when an element or layer is referred to as being “directly on,” “directly connected to,” or “directly coupled to” another element or layer, there are no intervening elements or layers present. The same applies to other expressions for describing a relationship between elements.

Unless otherwise defined herein, all terms including technical or scientific terms used herein have the same meanings as commonly understood by those skilled in the art to which the present invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

A combined reflectance confocal and two-photon microscopy system for non-invasive high-speed/high-contrast examination of living tissue according to the present invention relates to an apparatus that can provide images of an accurate cell structure by providing both cell and extracellular matrix contrasts through multi-imaging with reflection, extrinsic and intrinsic fluorescence, and second harmonic generation.

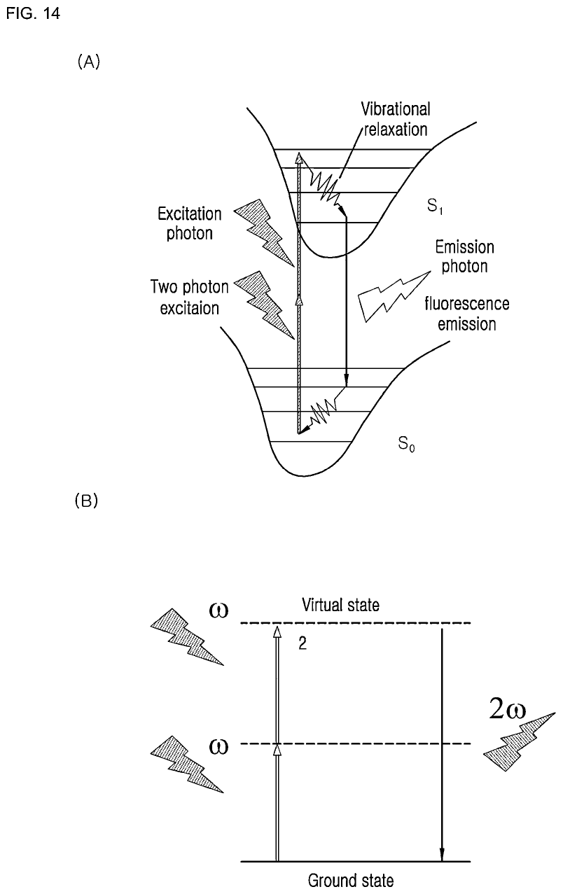

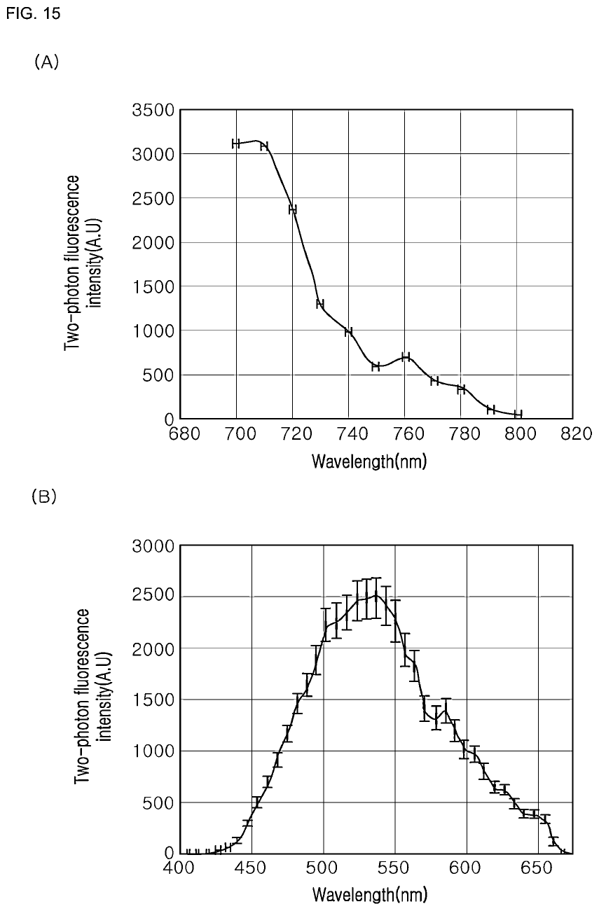

Prior to description of the microscopy system according to the present invention, a mechanism of excitation fluorescence of moxifloxacin and fluorescence excitation/emission spectrum will be described with reference to FIG. 14 and FIG. 15.

FIG. 14(A) is a view illustrating a mechanism of two-photon excitation fluorescence in the combined reflectance confocal and two-photon microscopy system for non-invasive high-speed/high-contrast examination of living tissue according to the present invention and the method for non-invasive high-speed/high-contrast examination of living tissue using the same.

The combined reflectance confocal and two-photon microscopy system for non-invasive high-speed/high-contrast examination of living tissue according to the present invention includes a light source 100 emitting laser beams and employs a femtosecond laser in order to obtain two-photon fluorescence, second harmonic generation, and reflected signals at the same time.

Here, for realization of two-photon fluorescence of moxifloxacin, first, an electron energy level of a fluorescent material molecule is raised from a ground state to an excited state using two excitation photons, as shown in FIG. 14(A).

Then, when the energy level of an electron drops again from the excited state to the ground state, a fluorescence photon is emitted. A phenomenon in which two excitation photons are absorbed and a single fluorescence photon is emitted as shown in FIG. 14(A) is referred to as two-photon excitation fluorescence.

That is, activity of molecules, cells, and tissues of an organism can be observed with high resolution through an optical fluorescence microscope when the molecules, cells, and tissues are treated with a fluorescent material. This is because an electron in the fluorescent material emits a fluorescent photon of a unique color in the course of being excited by an excitation photon and then returning to an original state thereof.

When the fluorescent material is injected into living tissue and is absorbed into cells of the living tissue to be maintained at high concentration, high contrast imaging of the living tissue is allowed using fluorescence of the fluorescent material.

When the fluorescent material staining on the living tissue is not toxic to the human body and fluorescence excitation is allowed in the visible light band that does not negatively affect the human body, it is possible to provide morphological information on the living tissue by staining the living tissue with the fluorescent material.

According to the present invention, fluoroquinolone antibiotics used for staining living tissue include moxifloxacin, gatifloxacin, pefloxacin, difloxacin, norfloxacin, ciprofloxacin, ofloxacin, enrofloxacin, and the like, and the living tissue is stained with moxifloxacin capable of exhibiting fluorescence in the visible band.

FIG. 14(B) is a view illustrating a mechanism of generating second harmonic signals in the combined reflectance confocal and two-photon microscopy system for non-invasive high-speed/high-contrast examination of living tissue according to the present invention and the method for non-invasive high-speed/high-contrast examination of living tissue using the same.

For generation of second harmonic signals, the energy level is raised from the ground state to a virtual state through a nonlinear process using two identical excitation photons, as shown in FIG. 14(B).

Then, when the energy level drops again from the virtual state to the ground state, a new photon with twice the energy of an initial excitation photon is emitted. A phenomenon in which a new photon with twice the energy of an initial photon is emitted through interaction of two photons with a non-linear material as shown in FIG. 14(B) is referred to as second harmonics.

FIG. 15(A) is a view of a two-photon florescence-based excitation spectrum in the combined reflectance confocal and two-photon microscopy system for non-invasive high-speed/high-contrast examination of living tissue according to the present invention and the method for non-invasive high-speed/high-contrast examination of living tissue using the same. Here, excitation light includes not only visible light in the wavelength band of 700 nm to 780 nm but also near-infrared light in the wavelength band of 780 nm to 850 nm.

0.5% (Alcon, USA) Vigamox eye drops commercially available in the art were used as moxifloxacin used in a living tissue imaging method using fluoroquinolone antibiotics according to the present invention.

FIG. 15(B) is a view of a fluorescence emission spectrum in the combined reflectance confocal and two-photon microscopy system for non-invasive high-speed/high-contrast examination of living tissue according to the present invention and the method for non-invasive high-speed/high-contrast examination of living tissue using the same. It could be seen that fluorescence emission was efficiently obtained at a wavelength of 450 nm or more.

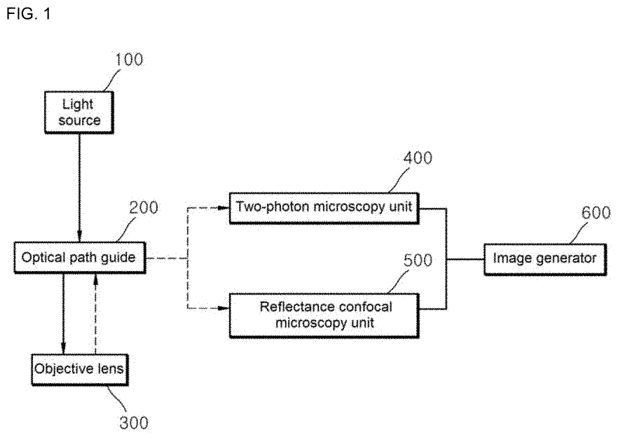

Hereinafter, the combined reflectance confocal and two-photon microscopy system for non-invasive high-speed/high-contrast examination of living tissue and a method for non-invasive high-speed/high-contrast examination of living tissue using the same will be described with reference to FIG. 1 to FIG. 13.

FIG. 1 is a block diagram of a combined reflectance confocal and two-photon microscopy system for non-invasive high-speed/high-contrast examination of living tissue according to the present invention.

Referring to FIG. 1, the combined reflectance confocal and two-photon microscopy system for non-invasive high-speed/high-contrast examination of living tissue according to the present invention includes a light source 100, an objective lens 300, a two-photon microscopy unit 400, a reflectance confocal microscopy unit 500, an optical path guide 200, and an image generator 600.

The light source 100 may emit laser beams to living tissue and may be composed of a femtosecond laser.

The living tissue to be irradiated with the laser beams is stained with FDA-approved moxifloxacin antibiotics as a cell staining material, whereby high-contrast cell images and information on an extracellular matrix including collagen (cell: moxifloxacin fluorescence, extracellular matrix: auto-fluorescence and second harmonics) can be specifically provided. Details of these features will be described below.

The objective lens 300 delivers light received from the light source 100 to the living tissue and may be coupled to a piezoelectric objective translator to adjust a focus with respect to the living tissue in a depth direction (perpendicular direction) through movement in an axial direction.

The two-photon microscopy unit 400 generates images of the cells and the extracellular matrix in the living tissue by photographing with effect light reflected from the living tissue and subjected to fluorescence excitation through moxifloxacin. Details of the two-photon microscopy unit 400 will be described below.

The reflectance confocal microscopy unit 500 generates images of an internal structure of the living tissue by photographing with effect light reflected from the living tissue. Details of the reflectance confocal microscopy unit 500 will be described below.

The optical path guide 200 provides a traveling path of the laser beam from the light source 100 to the objective lens 300 and guides the effect light reflected from the living tissue and excited by moxifloxacin to the two-photon microscopy unit 400 and the reflectance confocal microscopy unit 500. Details of the optical path guide 200 will be described below.

The image generator 600 generates images of the living tissue photographed by the two-photon microscopy unit 400 and the reflectance confocal microscopy unit 500. Details of the image generator 600 will be described below.

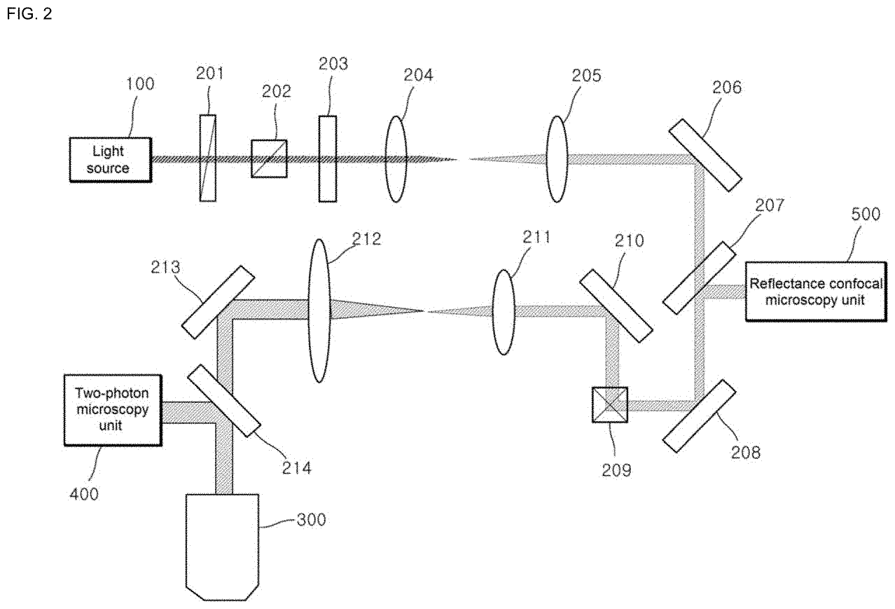

FIG. 2 is a schematic view of the optical path guide 200 of the combined reflectance confocal and two-photon microscopy system for non-invasive high-speed/high-contrast examination of living tissue according to the present invention.

Referring to FIG. 2, the combined reflectance confocal and two-photon microscopy system according to the present invention may include a half-wave plate (HWP) 201, a polarizing plate 202, a first optical filter 203, a first lens group, a first mirror 206, a beam splitter 207, a second mirror 208, a scanner 209, a third mirror 210, a second lens group, a fourth mirror 213, and a first dichroic mirror 214, in which components of the two-photon microscopy unit 400 are sequentially arranged in the stated order.

The half-wave plate 201 and the polarizing plate 202 adjust power of laser beams emitted from the light source 100 to produce polarized light.

The first optical filter 203 allows a laser beam having a wavelength of 680 nm or more to pass therethrough among the laser beams received from the polarizing plate 202.

The first lens group includes a first lens 204 and a second lens 205 and expands the laser beam received from the first optical filter 203.

The first mirror 206 reflects the laser beam received from the first lens group towards the beam splitter 207.

The beam splitter 207 allows the laser beam traveling from the first mirror 206 to the objective lens 300 to pass therethrough and guides effect light to the reflectance confocal microscopy unit 500.

The scanner 209 guides the laser beam received from the beam splitter 207 to the second mirror 208 while moving a focus of the laser beam on the living tissue on vertical/horizontal directions through sweeping. The scanner 209 may be composed of a combination of a galvanometer scanner and a resonance scanner.

The third mirror 210 guides the laser beam received from the scanner 209 to the second lens group.

The second lens group includes a third lens 211 and a fourth lens 212 and expands the laser beam received from the third mirror 210.

The fourth mirror 213 guides the laser beam received from the second lens group to the first dichroic mirror 214.

The first dichroic mirror 214 allows the laser beam traveling from the fourth mirror 213 to the objective lens 300 to pass therethrough and guides the effect light x to the two-photon microscopy unit 400 and the reflectance confocal microscopy unit 500.

The laser beam having passed through first dichroic mirror 214 is delivered to the living tissue through the objective lens 300.

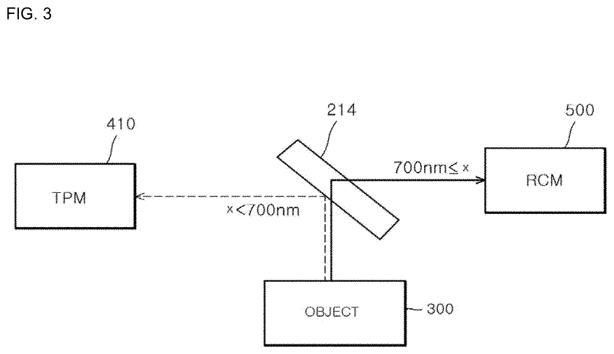

FIG. 3 is a schematic view of an optical path guided by the first dichroic mirror 214 in the optical path guide 200 of the combined reflectance confocal and two-photon microscopy system for non-invasive high-speed/high-contrast examination of living tissue according to the present invention.

The first dichroic mirror 214 splits the effect light reflected from and excited by the living tissue, in which the effect light includes first effect light excited by the living tissue and second effect light reflected from the living tissue.

Referring to FIG. 3, the first dichroic mirror 214 reflects the first effect light having a wavelength of less than 700 nm to be guided to the two-photon microscopy unit 400 and allows the second effect light having a wavelength of 700 nm or more to pass therethrough and to be guided to the reflectance confocal microscopy unit 500.

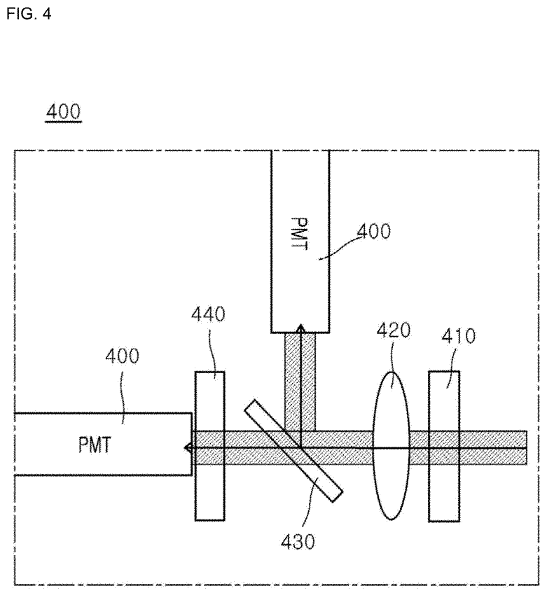

FIG. 4 is a schematic view of the two-photon microscopy unit 400 of the combined reflectance confocal and two-photon microscopy system for non-invasive high-speed/high-contrast examination of living tissue according to the present invention, showing a process and components for splitting and guiding the first effect light in the two-photon microscopy unit 400.

Referring to FIG. 4, the two-photon microscopy unit 400 includes a second optical filter 410, a fifth lens 420, a second dichroic mirror 430, a third optical filter 440, a first photomultiplier tube 450, and a second photomultiplier tube 460.

The second optical filter 410 allows light having a wavelength of less than 680 nm among the first effect light to pass therethrough.

The fifth lens 420 adjusts a focus of the first effect light having passed through the second optical filter 410.

The second dichroic mirror 430 allows a first-1 effect light component having a wavelength of less than 450 nm to pass therethrough and reflects a first-2 effect light component having a wavelength of 450 nm or more, among the first effect light received from the fifth lens 420.

The third optical filter 440 allows a first-1a effect light component having a wavelength of 390 nm to less than 410 nm to pass therethrough among the first-1 effect light component having passed through the second dichroic mirror 430.

The first photomultiplier tube 450 collects the first-1 a effect light component and the second photomultiplier tube 460 collects the first-2 effect light component.

Moxifloxacin fluorescence and second harmonics are separately collected from the first-la effect light component and the first-2 effect light component collected by the first photomultiplier tube 450 and the second photomultiplier tube 460, respectively, whereby signals of the first photomultiplier tube 450 and the second photomultiplier tube 460 can be supplied to an impedance amplifier and an information collection board.

The two-photon microscopy unit 400 specifically provides high-contrast cell images and information on the extracellular matrix including collagen (cell: moxifloxacin fluorescence, extracellular matrix: auto-fluorescence and second harmonics), as obtained from living tissue stained with FDA-approved moxifloxacin antibiotics as a cell staining material, to an existing microscope providing two-photon florescence-based auto-fluorescence and second harmonics information.

By the aforementioned features of the two-photon microscopy unit 400, it is possible to achieve detection of cancer cells in the dermis, which is not allowed by the reflectance confocal microscopy unit 500.

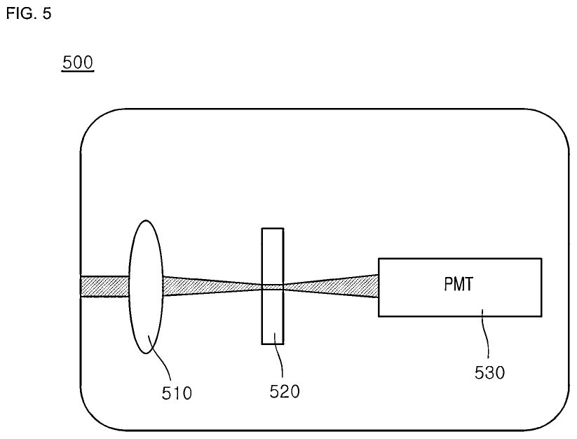

FIG. 5 is a schematic view of the reflectance confocal microscopy unit 500 of the combined reflectance confocal and two-photon microscopy system for non-invasive high-speed/high-contrast examination of living tissue according to the present invention, showing a process and components for guiding the second effect light in the reflectance confocal microscopy unit.

After passing through the first dichroic mirror 214, the second effect light sequentially passes through the fourth mirror 213, the second lens group, the third mirror 210, the scanner 209 and the second mirror 208, and is guided to the reflectance confocal microscopy unit 500.

Referring to FIG. 5, the reflectance confocal microscopy unit 500 includes a focus adjusting lens 510, a pin-hole 520, and a third photomultiplier tube 530.

The third photomultiplier tube 510 adjusts a focus of the second effect light having passed through the first dichroic mirror 214, and the pin-hole 520 has a diameter of 15 μm and allows the second effect light to pass therethrough such that the second effect light having passed through the pin-hole 520 is collected by the third photomultiplier tube 530.

The image generator 600 generates images of the first to third effect light, which is collected by the first to third photomultiplier tubes 450, 460, 530 and supplied to the impedance amplifier and the information collection board, using scan image software. Here, noise is removed from the images through post-processing by a block matching 3D optical filter in MATLAB.

The reflectance confocal microscopy unit 500 provides information on a fine structure of the living tissue through reflected signals in a reflected signal-based photographing manner.

For example, cells in the skin have high reflectivity and can be imaged due to higher refractive index (RI) of cell nuclei than those of the surrounding cytoplasm. Here, all constituents of tissue, such as cells and extracellular matrix, generate reflected signals, thereby providing morphology information alone without specifically providing information on the cells or extracellular matrix.

That is, in normal skin, cells of the epithelial layer, an extracellular matrix of the dermis, and blood vessels can be imaged. However, when deformation of the tissue structure occurs like cancer, cell clusters exhibit strong reflectivity to provide small difference in reflected signal with the extracellular matrix, thereby causing reduction in contrast.

Accordingly, although it is difficult to detect cancer in the dermis using an image provided from the reflectance confocal microscopy unit 500, the reflectance confocal microscopy unit 500 provides an image of a cell layer on the surface of intact skin, which can be useful for detection of cancer spreading on the epidermis.

As a result, since the images of the living tissue photographed by the two-photon microscopy unit 400 and the reflectance confocal microscopy unit 500 can be compared at the same time, it is possible to obtain more accurate information on the state of cells. Details of this effect will be described below.

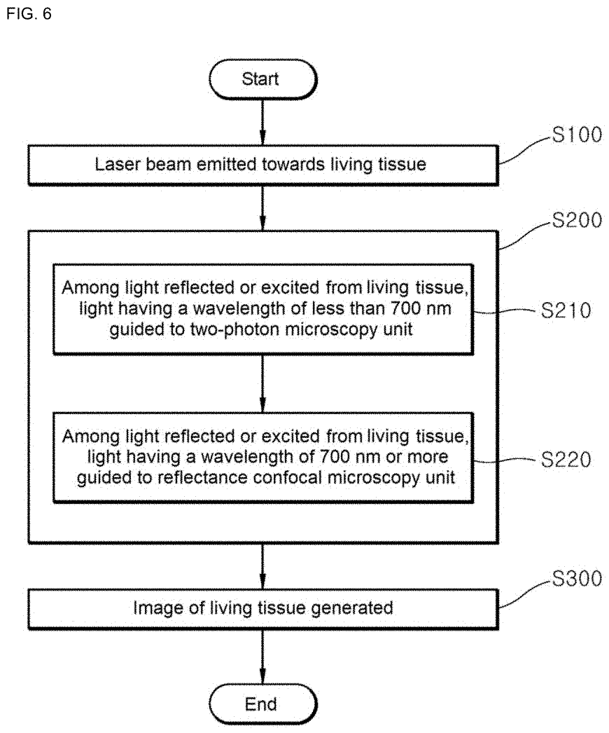

FIG. 6 is a flowchart of a non-invasive high-speed/high-contrast examination method using the combined reflectance confocal and two-photon microscopy system for non-invasive high-speed/high-contrast examination of living tissue according to the present invention.

Referring to FIG. 6, the non-invasive high-speed/high-contrast examination method using a combined reflectance confocal and two-photon microscopy system for non-invasive high-speed/high-contrast examination of living tissue according to the present invention includes a living tissue staining step, an irradiation step S100, an effect light guide step S200, and an image generation step S300.

The non-invasive high-speed/high-contrast examination method using the combined reflectance confocal and two-photon microscopy system according to the present invention is a rapid non-invasive examination method based on complementary information (reflection, fluorescence-based cells, second harmonic signal-based extracellular matrix) and three-dimensional resolution through combination of existing reflectance confocal microscopy and two-photon microscopy using moxifloxacin, that is, a fluoroquinolone antibiotic, as a fluorescent cell staining material. Each step of the non-invasive high-speed/high-contrast examination method will now be described.

In the living tissue staining step of the non-invasive high-speed/high-contrast examination method, living tissue provided as an examination target is stained.

Here, living tissue to be irradiated with laser beams is stained with FDA-approved moxifloxacin antibiotics as a cell staining material, whereby high-contrast cell images and information on an extracellular matrix including collagen (cell: moxifloxacin fluorescence, extracellular matrix: auto-fluorescence and second harmonics) can be specifically provided.

Moxifloxacin is an antibiotic currently used to treat or prevent bacterial infection in clinical practice. Moxifloxacin has intrinsic fluorescence, exhibits excellent tissue permeability to stain living tissue, and has advantageous properties for fluorescence imaging. Moxifloxacin is commercially available not only as eye drops but also as oral preparations and vascular injections for prevention and treatment of keratitis.

In the irradiation step S100, the light source 100 emits laser beams towards the living tissue.

In the effect light guide step S200, effect light reflected from the living tissue is allowed to travel to the two-photon microscopy unit 400 and the reflectance confocal microscopy unit 500.

In the image generation step S300, images of the living tissue photographed by the two-photon microscopy unit 400 and the reflectance confocal microscopy unit 500 are generated by the image generator 600.

Here, the effect light guide step S200 includes a first effect light guide step S210 and a second effect light guide step S220.

In the first effect light guide step S210, first effect light having a wavelength of less than 700 nm is reflected and guided to the two-photon microscopy unit 400 among the effect light reflected from the living tissue and subjected to fluorescence excitation.

In the second effect light guide step S220, among the effect light reflected from the living tissue, second effect light having a wavelength of 700 nm or more is allowed to pass through the first dichroic mirror and is guided to the reflectance confocal microscopy unit 500.

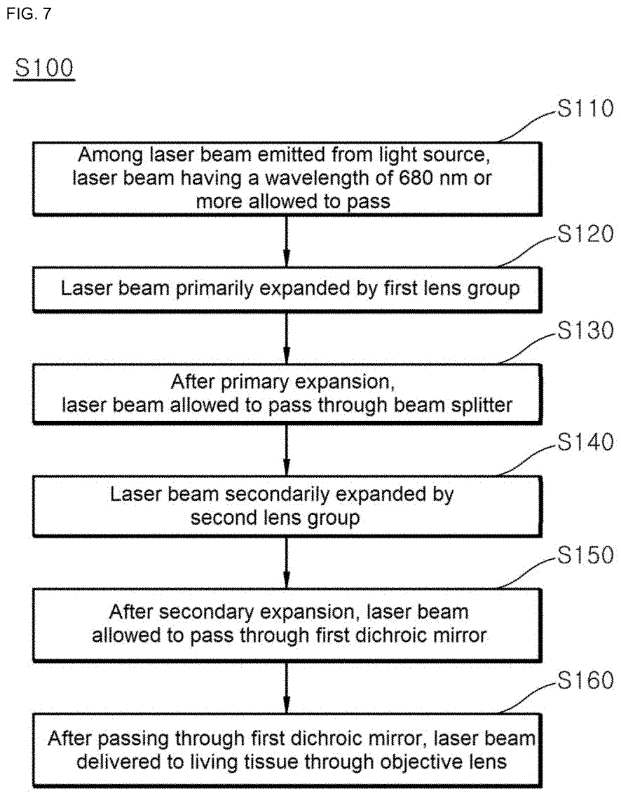

FIG. 7 is a flowchart of the irradiation step S100 in the non-invasive high-speed/high-contrast examination method using the combined reflectance confocal and two-photon microscopy system for non-invasive high-speed/high-contrast examination of living tissue according to the present invention.

Referring to FIG. 7, the irradiation step S100 includes a first light filtering step S110, a first beam expansion step S120, a first beam passing step S130, a second beam expansion step S140, a second beam passing step S150, and a living tissue irradiation step S160.

In the first light filtering step S110, only a laser beam having a wavelength of 680 nm or more among the laser beams emitted from the light source 100 is allowed to pass through the first optical filter.

In the first beam expansion step S120, the first lens group primarily expands the laser beam.

In the first beam passing step S130, the laser beam subjected to primary expansion is allowed to pass through the beam splitter 207, and in the second beam expansion step S140, the second lens group secondarily expands the laser beam.

In the second beam passing step S150, the laser beam subjected to secondary expansion is allowed to pass through the first dichroic mirror 214, and in the living tissue irradiation step S160, the laser beam having passed through the first dichroic mirror 214 is delivered to the living tissue through the objective lens 300.

Next, in the non-invasive high-speed/high-contrast examination method using the combined reflectance confocal and two-photon microscopy system for non-invasive high-speed/high-contrast examination of living tissue according to the present invention, traveling of light reflected from the living tissue and excited by moxifloxacin in the two-photon microscopy unit 400 and the reflectance confocal microscopy unit 500 will be described.

Here, the two-photon microscopy unit 400 based on moxifloxacin fluorescence can perform high-speed imaging at high cell fluorescent contrast and can produce an image of the extra-cellular matrix (ECM) of the living tissue through intrinsic second harmonic generation (SHG).

In addition, the reflectance confocal microscopy unit 500 obtains reflected light reflected from the living tissue, thereby allowing combination of an existing reflectance confocal microscope and the two-photon microscopy unit 400.

Combination of the two-photon microscopy unit 400 and the reflectance confocal microscopy unit 500 enables high-speed multi-contrast imaging through a resonant scanner and a high-speed photon multiplier tube, and can overcome limitations of the existing confocal microscope through multi-imaging with reflection, fluorescence, and second harmonic contrast.

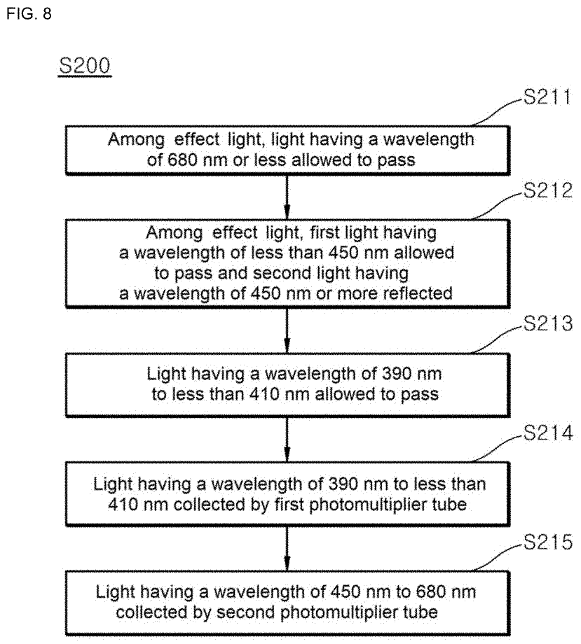

FIG. 8 is a flowchart of the first effect light guide step S210 in the non-invasive high-speed/high-contrast examination method using the combined reflectance confocal and two-photon microscopy system for non-invasive high-speed/high-contrast examination of living tissue according to the present invention, showing a traveling path of light in the two-photon microscopy unit 400.

Referring to FIG. 8, the first effect light guide step S210 includes a second light filtering step S211, a light distinguishing step, a third light filtering step S213, a first light collecting step S214, and a second light collecting step S215.

In the second light filtering step S211, among the effect light, light having a wavelength of less than 680 nm is allowed to pass through the second optical filter, and in the light distinguishing step, among the effect light, first-1 effect light having a wavelength of less than 450 nm is allowed to pass through the second dichroic mirror and first-2 effect light having a wavelength of 450 nm or more is reflected thereby.

In the third light filtering step S213, first-1a effect light having a wavelength of 390 nm to less than 410 nm among the first-1 effect light is allowed to pass through the third optical filter.

In the first light collecting step S214, the first-la effect light component is collected by the first photomultiplier tube 450 and, in the second light collecting step S215, the first-2 effect light component is collected by the second photomultiplier tube 460.

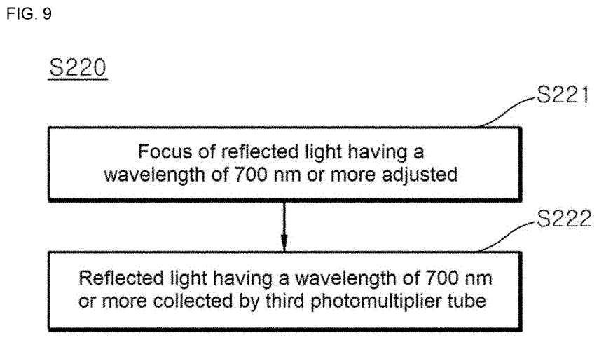

FIG. 9 is a flowchart of the second effect light guide step S220 in the non-invasive high-speed/high-contrast examination method using the combined reflectance confocal and two-photon microscopy system for non-invasive high-speed/high-contrast examination of living tissue according to the present invention, showing a traveling path of light in the reflectance confocal microscopy unit 500.

Referring to FIG. 9, the second effect light guide step S220 includes a focus adjusting step S221 in which a focus of the second effect light is adjusted, and a third light collecting step S222 in which the second effect light is collected by the third photomultiplier tube 530.

As described above, the combined reflectance confocal and two-photon microscopy system for non-invasive high-speed/high-contrast examination of living tissue according to the present invention and the method for non-invasive high-speed/high-contrast examination of living tissue using the same can provide extracellular matrix/cell contrast through multi-imaging with reflection, fluorescence, and second harmonic contrast, and enables accurate imaging of a cell structure by simultaneously providing information on a cell membrane (reflected signal) and cytoplasm (fluorescence signal) of epidermal cells upon in-vivo photographing of living tissue using the two-photon microscopy unit 400 and the reflectance confocal microscopy unit 500.

Consequently, in the non-invasive high-speed/high-contrast examination method using the combined reflectance confocal and two-photon microscopy system for non-invasive high-speed/high-contrast examination of living tissue according to the present invention, cells are stained with moxifloxacin, that is, a fluoroquinolone antibiotic, by dropping moxifloxacin onto living tissue, which in turn is simultaneously photographed by the reflectance confocal microscopy unit 500 and the moxifloxacin-based two-photon microscopy unit 400 using light having a wavelength of 700 nm to 850 nm as excitation light. Here, reflection information in the wavelength range of 700 nm to 850 nm, moxifloxacin fluorescence information in the range of 400 nm to 650 nm, and second harmonic information in the range of 380 nm to 450 nm can be simultaneously obtained in multi-contrast images.

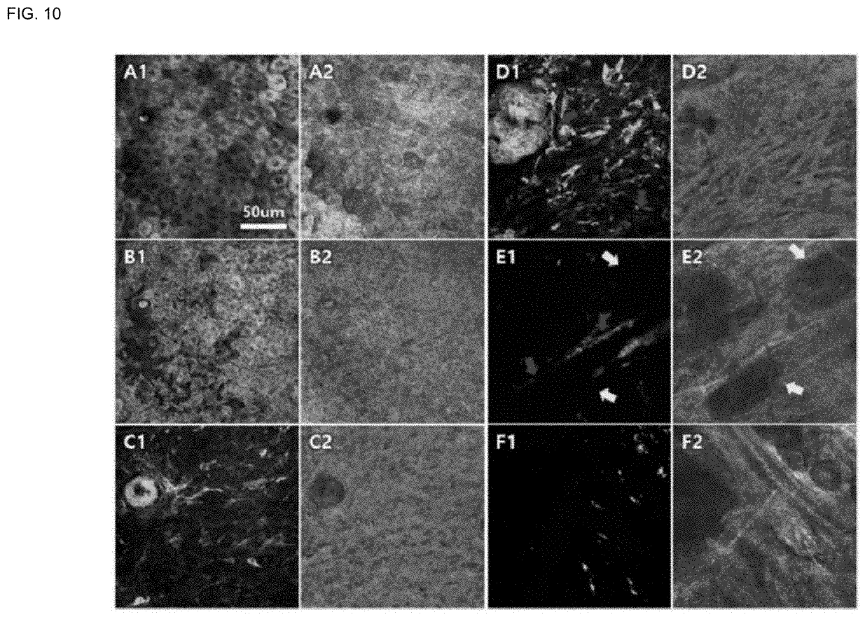

Referring to FIG. 10 to FIG. 13, the following description will focus on information on living tissue that can be confirmed through images obtained through the combined reflectance confocal and two-photon microscopy system for non-invasive high-speed/high-contrast examination of living tissue according to the present invention and the method for non-invasive high-speed/high-contrast examination of living tissue using the same.

FIG. 10 is two-photon and reflection confocal images of the skin of a mouse ear stained with moxifloxacin, as obtained through photographing by the combined reflectance confocal and two-photon microscopy system for non-invasive high-speed/high-contrast examination of living tissue according to the present invention and the method for non-invasive high-speed/high-contrast examination of living tissue using the same.

FIG. 10 shows images photographed at six depths of 4 μm (A1, A2), 16 μm (B1, B2), 24 μm (C1, C2), 41 μm (D1, D2), 64 μm (E1, E2), and 105 μm (F1, F2) from the skin surface, in which images A1 to F1 were photographed after moxifloxacin excitation by the two-photon microscopy unit 400 and images A2 to F2 were photographed by the reflectance confocal microscopy unit 500.

A1 and A2 correspond to the stratum corneum, B1 and B2 correspond to the basal layer, and C1, C2, D1, D2, E1, E2, F1, and F2 correspond to the dermis at three different depths.

The images photographed by the two-photon microscopy unit 400 are green and blue-scale images for moxifloxacin fluorescence and second harmonics, respectively; the images photographed by the reflectance confocal microscopy unit 500 are grayscale images; and the blood and lymphatic vessels of the dermis are indicated by red and yellow arrows, respectively.

Referring to FIG. 10, in the non-invasive high-speed/high-contrast examination method using the combined reflectance confocal and two-photon microscopy system according to the present invention, an accurate structure of cells was photographed by simultaneously providing information on the cell membrane (reflected signal) and cytoplasm (fluorescence signal) of the epidermal cells when moxifloxacin was dropped onto normal skin of a mouse and in-vivo photographing of the normal skin was performed using a combined system of the reflectance confocal microscopy unit 500 and the moxifloxacin-based two-photon microscopy unit 400.

In a relatively deep dermal layer, the structures of the cell membrane and the extracellular matrix were photographed based on the reflected signals, and at the same time, the fluorescence/second harmonic signals provided additional information on distribution of collagen in the cells and the matrix to show the skin microenvironment with high contrast.

That is, in the epidermis (stratum corneum, basal layer), epidermal cells are observed in both images photographed by the two-photon microscopy unit 400 and the reflectance confocal microscopy unit 500, and it can be seen that the epidermal cells are large and flat in the stratum corneum and small in the basal layer.

In addition, among the images A1 and A2 of FIG. 10, the image photographed by the two-photon microscopy unit 400 shows individual cells decomposed in cytoplasm by high moxifloxacin fluorescence and an accurate structure of the epidermal cells by providing high-fluorescence information on cytoplasm and low-fluorescence information on the cell nucleus; and the image photographed by the reflectance confocal microscopy unit 500 shows individual cells decomposed in the cell membrane to be photographed at relatively high reflectivity.

Further, among the images C1 and C2 of FIG. 10, the image photographed by the two-photon microscopy unit 400 provides information on dermal cells and distribution of collagen, and the image photographed by the reflectance confocal microscopy unit 500 corresponds to the structure of the cell membrane and the extracellular matrix. Thus, the two-photon microscopy unit 400 and the reflectance confocal microscopy unit 500 provide different information, thereby enabling accurate visualization of the skin microenvironment through combination of the images photographed by the two-photon microscopy unit 400 and the reflectance confocal microscopy unit 500.

Specifically, dermal cells and collagen in the dermis can be observed in the images photographed by the two-photon microscopy unit 400; and the structure of a fibrous extracellular matrix (extracellular matrix) and the blood vessels can be observed in the images photographed by the reflectance confocal microscopy unit 500 (see C1, C2, D1, and D2 of FIG. 10). Here, the cells distributed in the dermis are indicated by moxifloxacin labels in the images photographed by the two-photon microscopy unit 400 and are not observed in the images photographed by the reflectance confocal microscopy unit 500.

Cell clusters of the hair follicle can be observed in the images photographed by the two-photon microscopy unit 400 and distribution of collagen in the extracellular matrix is visualized in a second harmonic channel of the two-photon microscopy unit 400.

The structure of the fibrous extracellular matrix can be clearly observed in the images photographed by the reflectance confocal microscopy unit 500. In the extracellular matrix, a relatively large fibrous structure and the content of collagen are visualized through combination of the second harmonic channel and the image photographed by the reflectance confocal microscopy unit 500.

The blood vessels can be observed in all of the images photographed by the two-photon microscopy unit 400 and the reflectance confocal microscopy unit 500 (see red arrows in D1, D2, E1, and E2 of FIG. 10). As shown in the images photographed by the reflectance confocal microscopy unit 500, the blood vessels exhibit relatively strong and weak reflection on upper surfaces thereof and can be partially observed together with moxifloxacin marks of endothelial cells in the images photographed by the two-photon microscopy unit 400.

Large blood vessels deep in the skin can be observed only in the image photographed by the reflectance confocal microscopy unit 500 (see a red arrow of F2 in FIG. 10) due to insufficient infiltration of moxifloxacin thereinto and very weak second harmonic signals caused by scattering of excitation light at this depth.

All of the images photographed by the two-photon microscopy unit 400 and the reflectance confocal microscopy unit 500 show an empty structure (see red arrows of E1 and E2 in FIG. 10), which may be a lymphatic vessel, and the cell structure of the epithelium can be observed in all of the images photographed by the two-photon microscopy unit 400 and the reflectance confocal microscopy unit 500.

The dermal cells and collagen in the dermis were visualized by the two-photon microscopy unit 400; the structure of the fibrous extracellular matrix and the blood vessels were visualized by the reflectance confocal microscopy unit 500, and a combination of the images photographed by the two-photon microscopy unit 400 and the reflectance confocal microscopy unit 500 allowed accurate visualization of the skin microenvironment by imaging the cells, the extracellular matrix, the blood vessels and the lymphatic vessels.

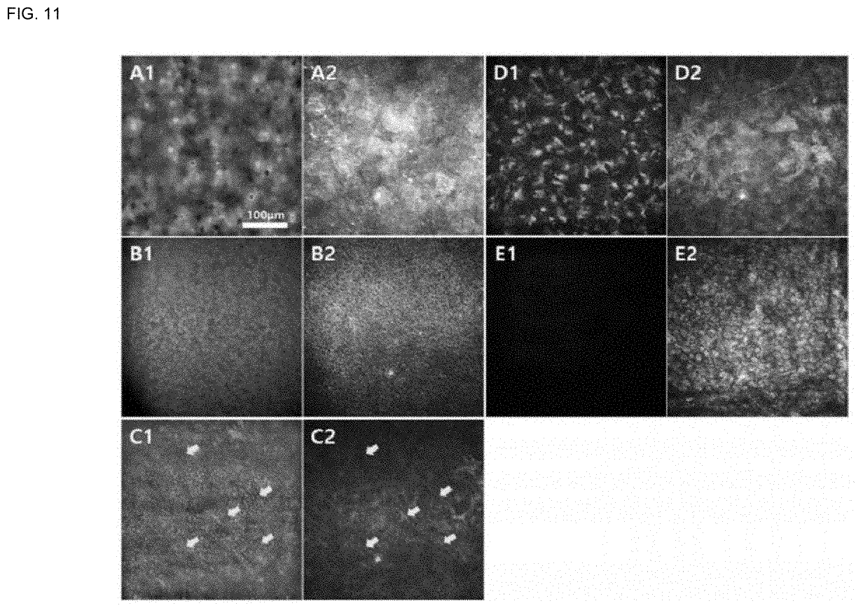

FIG. 11 is two-photon and reflection confocal images of the skin of a mouse ear stained with moxifloxacin, as obtained through photographing by the combined reflectance confocal and two-photon microscopy system for non-invasive high-speed/high-contrast examination of living tissue according to the present invention.

FIG. 11 shows images photographed at five depths of 3 μm (A1, A2), 15 μm (B1, B2), 20 μm (C1, C2), 33 μm (D1, D2), and 65 μm (E1, E2) from the skin surface, in which images A1 to E1 were photographed after moxifloxacin excitation by the two-photon microscopy unit 400 and images A2 to E2 were photographed by the reflectance confocal microscopy unit 500.

Further, A1 and A2 correspond to the superficial epithelium, B1 and B2 correspond to the basal epithelium, C1 and C2 correspond to the basal nerve layer (indicated by red arrows), D1 and D2 correspond to the stroma including nerves (indicated by red arrows), and E1 and E2 correspond to the endothelium.

The images A1 to E1 photographed by the two-photon microscopy unit 400 are green and blue-scale images for moxifloxacin fluorescence and second harmonics, respectively, and the images A2 to E2 photographed by the reflectance confocal microscopy unit 500 are grayscale images.

The combined reflectance confocal and two-photon microscopy system for non-invasive high-speed/high-contrast examination of living tissue according to the present invention was applied to the cornea of a normal mouse in an in-vivo state to perform early non-invasive diagnosis of corneal infection. Results are as follows.

The images photographed by the two-photon microscopy unit 400 and the reflectance confocal microscopy unit 500 show cells and extracellular matrix in the epithelium (see A1, A2, B1, B2, C1, and C2 of FIG. 11), the stroma (see D1 and D2 of FIG. 11), and the endothelium (see E1 and E2 of FIG. 11) of the cornea of the mouse.

In the epithelium, relatively large and flat cells of the superficial epithelium (see A1 and A2 of FIG. 11) and relatively small cells of the basal epithelium (see B1, B2, C1 and C2 of FIG. 11) can be observed in all of the images photographed by the two-photon microscopy unit 400 and the reflectance confocal microscopy unit 500.

In the image photographed by the two-photon microscopy unit 400, it can be confirmed that the epithelium cells exhibit relatively uniform moxifloxacin fluorescence in cytoplasm and individual cells are decomposed due to relatively weak fluorescence at the cell boundary and variation in intensity therebetween.

In the image photographed by the reflectance confocal microscopy unit 500, it can be confirmed that the cells are decomposed by relatively high reflectivity at the boundary between the cells due to difference in refractive index therebetween.

In addition, since cells of the basal epithelium have a smaller and longer size than cells of the superficial epithelium, decomposition of the cells of the basal epithelium can be more apparently confirmed than decomposition of the cells of the superficial epithelium. Although the sub-basal nerve plexuses are observed in both images photographed by the two-photon microscopy unit 400 and the reflectance confocal microscopy unit 500, the sub-basal nerve plexuses can be more clearly observed in the images photographed by the reflectance confocal microscopy unit 500 (see C1 and C2 of FIG. 11) due to high reflectivity resulting from difference in refractive index

In the stroma, cells including keratocytes, nerves, and collagen were visualized. The keratocytes and the nerves exhibit moxifloxacin fluorescence and relatively high reflectivity and can be observed in the images photographed by the two-photon microscopy unit 400 and the reflectance confocal microscopy unit 500, whereas collagen can be observed only in the images photographed by the two-photon microscopy unit 400 (see D1 and D2 of FIG. 11) through the second harmonics.

Further, the cells can be clearly observed in the images photographed at relatively high reflectivity by the reflectance confocal microscopy unit 500 and the cell nuclei can be observed in the images photographed by the two-photon microscopy unit 400.

In the endothelium, the cells are clearly observed only in the images photographed by the reflectance confocal microscopy unit 500 due to insufficient moxifloxacin labeling in the two-photon microscopy unit 400.

In the cornea of a normal mouse, cells of all corneal layers can be clearly observed in the images photographed by the reflectance confocal microscopy unit 500 and can also be observed in the images photographed by the two-photon microscopy unit 400.

Further, as described above, although the second harmonics can be used to check collagen in the stroma and most of the cell structure in the normal cornea can be sufficiently checked through comparison with the images photographed by the reflectance confocal microscopy unit 500, these features may be usefully used to visualize the cornea clouded by lesions through comparison of the cells and collagen in the corneal layer.

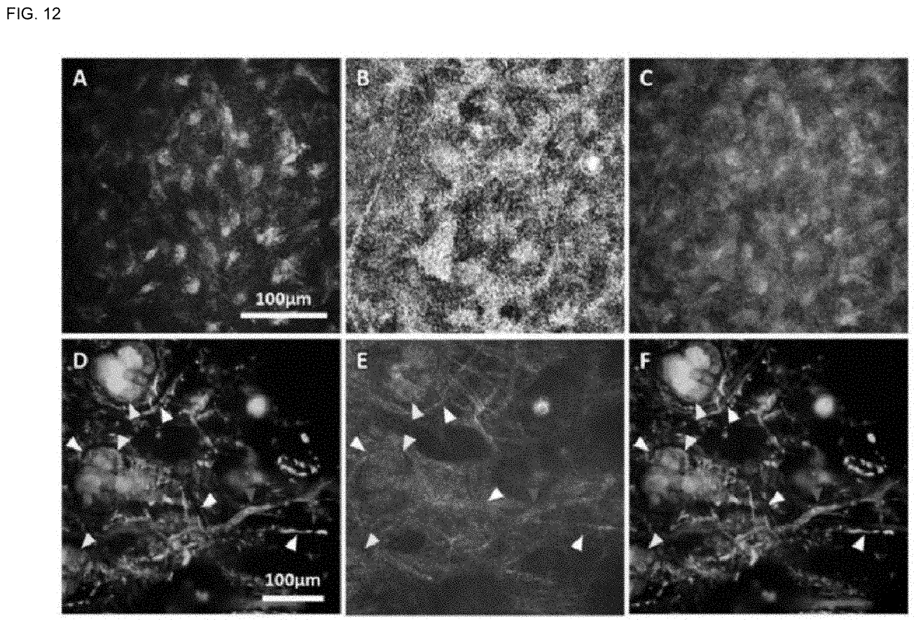

FIG. 12 is two-photon and reflection confocal images of the cornea and the skin of a mouse stained with moxifloxacin, as obtained through photographing by the combined reflectance confocal and two-photon microscopy system for non-invasive high-speed/high-contrast examination of living tissue according to the present invention.

In FIG. 12, A is an image of the corneal stroma photographed by the two-photon microscopy unit 400, B is an image of the corneal stroma photographed by the reflectance confocal microscopy unit 500, and C is a combined image of the image of the corneal stroma photographed by the two-photon microscopy unit 400 and the image of the corneal stroma photographed by the reflectance confocal microscopy unit 500.

Here, in the combined image C, the images of the corneal stroma photographed by the reflectance confocal microscopy unit 500 are colored in red scales in order to improve contrast of the images photographed by the two-photon microscopy unit and colored in green and blue.

The non-invasive high-speed/high-contrast examination method using the combined reflectance confocal and two-photon microscopy system for non-invasive high-speed/high-contrast examination of living tissue according to the present invention provides additional information on the cells and collagen through in-vivo multi-contrast images of the cornea as another living tissue.

The combined reflectance confocal and two-photon microscopy system for non-invasive high-speed/high-contrast examination of living tissue according to the present invention provides information on the cells of the corneal epithelium through reflected signals and fluorescence signals, and information on the cell nuclei and collagen distribution in the extracellular matrix of the corneal stroma through reflected signals and fluorescence/second harmonic signals. Additional comparison of the cells and the extracellular matrix may be useful for accurate detection and diagnosis of a causative agent of the cornea in disease, which makes it difficult to detect cells of the damaged cornea only through scattered signals, since the damaged cornea has a microstructure damaged by disease and is not transparent.

Specifically, A of FIG. 12 is an image of a stroma region of the cornea photographed by the two-photon microscopy unit 400 and shows the cell nuclei and collagen distribution in the extracellular matrix through variation in in-vivo distribution of moxifloxacin.

In addition, B of FIG. 12 is an image of the stroma region of the cornea simultaneously photographed with A of FIG. 12 by the reflectance confocal microscopy unit 500 and shows the overall shape of cells including cytoplasm based on strong reflected signals from the cell membrane. Here, collagen of the corneal stroma can be observed based on the second harmonics.

Further, C of FIG. 12 is a combined image of an image photographed by the two-photon microscopy unit 400 and an image photographed by the reflectance confocal microscopy unit 500, in which moxifloxacin fluorescence, second harmonics, and reflected signals are represented in green, blue and red to show detailed microenvironment through multi-contrast images.

Further, D is an image of the skin dermis photographed by the two-photon microscopy unit 400, E is an image of the skin dermis photographed by the reflectance confocal microscopy unit 500, and F is a combined image of the image of the skin dermis photographed by the two-photon microscopy unit 400 and an image of the skin dermis photographed by the reflectance confocal microscopy unit 500, which includes a blood flow image.

Here, in the combined image F, two-photon, second harmonics, and reflectance confocal regions are represented in green, blue and red for individual comparison.

Dermal cells, cell clusters (yellow arrow) in the hair follicle, endothelial cells (white arrow) in the blood vessel wall, and cells including nerves (purple arrow) can be clearly observed in D of FIG. 12, that is, in the image of the skin dermis photographed by the two-photon microscopy unit 400.

Further, distribution of the extracellular matrix containing the fibrous structure and the hair follicles depending on variation in reflectivity can be clearly observed in E of FIG. 12, that is, in the image of the skin dermis photographed by the reflectance confocal microscopy unit 500.

Further, the blood flow can be relatively clearly shown in the image of the corneal stroma photographed by the reflectance confocal microscopy unit 500, and the cells, the structure of the extracellular matrix and the blood flow can be observed in the combined image F of in FIG. 12.

It can be seen that the blood flow appears in high contrast through additional processing of temporal intensity change, and some blood flow is present around the hair follicle and other blood flow is present across the dermis. In addition, the detailed microenvironment of the skin dermis can be checked through combination of reflection confocal microscopy, second harmonics, and two-photon microscopy.

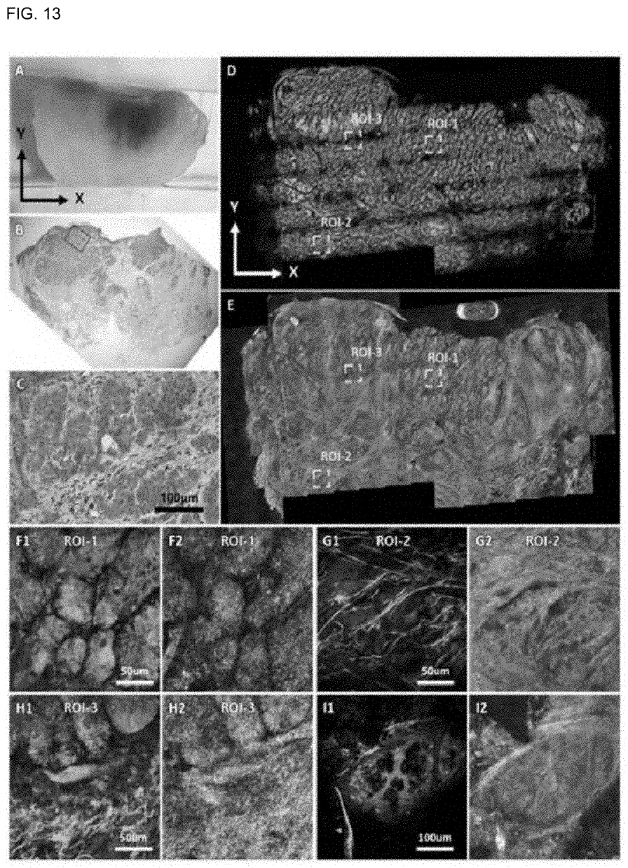

FIG. 13 is two-photon and reflection confocal images of human basal cell carcinoma (BCC) stained with moxifloxacin and a combined image thereof, as obtained through in-vivo photographing by the combined reflectance confocal and two-photon microscopy system for non-invasive high-speed/high-contrast examination of living tissue according to the present invention.

In FIG. 13, A is a bright field image of a basal cell carcinoma specimen; B and C are images stained with hematoxylin and eosin (hereinafter, H&E), in which C is an enlarged view of the area indicated by a black rectangular line in B; D is a cross-sectional mosaic image of basal cell carcinoma photographed by the two-photon microscopy unit 400, in which moxifloxacin fluorescence and second harmonics are distinguished with green and blue colors, respectively, and E is a cross-sectional mosaic image of basal cell carcinoma photographed by the reflectance confocal microscopy unit 500 and represented in grayscale.

F1, G1 and H1 are enlarged images of regions ROI-1, ROI-2, and ROI-3 in D of FIG. 13, and show cancer cells, an extracellular matrix, and the cells and the extracellular matrix, respectively, and F2, G2 and H2 are enlarged images of regions ROI-1, ROI-2 and ROI-3 in E of FIG. 13, and show cancer cells, an extracellular matrix, and the cells and the extracellular matrix, respectively.

In addition, I1 is an enlarged image of a region represented by a red dotted line in D of FIGS. 13 and 12 is an enlarged image of a region represented by a red dotted line in E of FIG. 13 and shows the sebaceous glands.

A micro-tissue environment of a lesion shows various differences from a normal environment, among which basal cell carcinoma, a type of skin cancer, has nest-shaped cancer cells as one characteristic and exhibits a relatively clear boundary with respect to normal tissue.

According to the non-invasive high-speed/high-contrast examination method using the combined reflectance confocal and two-photon microscopy system according to the present invention, images of basal cell carcinoma were obtained through ex-vivo photographing by the combined microscopy system of the two-photon microscopy unit 400 and the reflectance confocal microscopy unit 500, in which collagen distribution around the cancer cells and cancer cell clusters stained with moxifloxacin were photographed through high fluorescence signals.

These signals were provided simultaneously with reflected signals of the nest-shaped cancer cells to show the microstructure of basal cell carcinoma with high contrast. Here, in the images photographed by the reflectance confocal microscopy unit 500 through the reflected signals, a sebaceous gland region was not clearly distinguished from the nest-shaped cancer cells, whereas, in the images photographed by the two-photon microscopy unit 400 through the two-photon signals of moxifloxacin, an acinar structure like multi-leaf berries could be easily distinguished.

Specifically, a bright field image of a basal cell carcinoma specimen in A of FIG. 13 shows a partially colored skin specimen, in which an upper portion of the specimen including a colored region corresponds to basal cell carcinoma, and B and C of FIG. 13 are H&E stained histological images at two different fields of view (FOVs) and show distribution of cancer cells in the specimen.

In B of FIG. 13, a histological image at a high FOV shows basal cell carcinoma at an upper side and normal skin at a lower side, and in C of FIG. 13C, an enlarged histological image of inner basal cell carcinoma shows the structure of cancer clusters.

Combined cross-sectional images of cell carcinoma specimens and enlarged combined images in three regions of interest (ROIs) are shown in D to H of FIG. 13.

In FIGS. 13, D, F1, H1, G1, and I1 are images photographed by the two-photon microscopy unit 400, in which a moxifloxacin fluorescence signal and a collagen distribution signal are represented by green and blue colors, respectively, and E, F2, H2, G2, and I2 are images photographed by the reflectance confocal microscopy unit 500.

In D of FIG. 13, basal cell carcinoma is shown with a dense cell structure at a left upper end, in the middle and at the right side, and collagen distribution and a structure enriched with extracellular matrix, such as elastin, are shown at a left lower end, thereby indicating that two regions are clearly distinguished.

E of FIG. 13 is an image of the same region as D of FIG. 13 and shows cancer cell clusters of basal cell carcinoma and the structure of surrounding fibers. However, since these two regions are not clearly distinguished in E of FIG. 13 unlike D of FIG. 13, the image photographed by the two-photon microscopy unit 400 may act as a complementary image to solve this problem.

For detailed analysis, in each of D and E of FIG. 13, three regions-of-interest (ROI) were selected corresponding to inner cancer regions F1, F2, ROI-1, outer cancer regions G1, G2, ROI-2, and cancer boundary regions H1, H2, ROI-3.

As shown in F1 of FIG. 13, an image of the interior of a cancer region photographed by the two-photon microscopy unit 400 shows cancer cell clusters, in which the cancer cells are labelled with moxifloxacin. From F1 of FIG. 13, it can be seen that a small amount of collagen is present in the cancer cells.

As shown in F2 of FIG. 13, an image of the interior of the cancer region photographed by the reflectance confocal microscopy unit 500 more clearly shows the shape of the cancer cell clusters.

That is, both images photographed by the two-photon microscopy unit 400 and the reflectance confocal microscopy unit 500 show the cancer cell clusters of basal cell carcinoma, whereas only the image photographed by the two-photon microscopy unit 400 shows the fibrous structure.

G1 of FIG. 13 is an image of an outer region of cancer photographed by the two-photon microscopy unit 400 and shows collagen and elastin of a non-damaged extracellular matrix, and G2 of FIG. 13 G2 is an image of the outer region of cancer photographed by the reflectance confocal microscopy unit 500 and shows the fibrous structure.

In the images G1 and G2 of FIG. 13, the extracellular matrix is not damaged and the cancer cell clusters are not shown. Thus, cells in the outer regions can be classified as normal cells, as compared to the cancer cells present in the form of clusters.

In FIG. 13, H1 and H2 are images of cancer boundaries simultaneously photographed by the two-photon microscopy unit 400 and the reflectance confocal microscopy unit 500, in which the cancer cell clusters (upper region) are distinguished from an extracellular matrix region (lower region).

In FIG. 13, I1 is an image photographed by the two-photon microscopy unit 400, which clearly shows the acinar structure like multi-leaf berries such that the sebaceous glands can be clearly observed, and I2 is an image photographed by the reflectance confocal microscopy unit 500 in which the boundaries are shown similar to the cancer cell clusters, thereby making it difficult to achieve clear distinction from basal cancer cells.

Accordingly, it is possible to achieve more accurate diagnosis based on contemporary information obtained by checking the images simultaneously photographed by the two-photon microscopy unit 400 and the reflectance confocal microscopy unit 500.

Consequently, the combined reflectance confocal and two-photon microscopy system for non-invasive high-speed/high-contrast examination of living tissue according to the present invention provides more complete information with respect to the same region in the clinical environment by overlapping images of two channels.

Specifically, the combined reflectance confocal and two-photon microscopy system according to the present invention enables accurate detection of lesions by rapidly providing multi-contrast images (morphological information of reflection-based structures, moxifloxacin fluorescence-based cell images, and auto-fluorescence and second harmonics-based extracellular matrix images) in the clinical environment.

That is, since the FDA-approved antibiotic moxifloxacin exhibiting high tissue penetration and auto-fluorescence is used as a cell staining material, the two-photon microscopy unit 400 enabling high-speed imaging is combined with the reflectance confocal microscopy unit 500, whereby multi-contrast images for accurate analysis and diagnosis of lesions can be provided at high speed and applied to actual clinics.

Although some embodiments have been described herein, it should be understood that these embodiments are provided for illustration only and are not to be construed in any way as limiting the present invention, and that various modifications, changes, alterations, and equivalent embodiments can be made by those skilled in the art without departing from the spirit and scope of the invention. In addition, these modifications and the like are not to be regarded as a departure from the spirit and prospect of the present invention.

### LIST OF REFERENCE NUMERALS

100: Light source

200: Optical path guide

201: Half-wave plate

202: Polarizing plate

203: First optical filter

204: First lens

205: Second lens

206: First mirror

207: Beam splitter

208: Second mirror

209: Scanner

210: Third mirror

211: Third lens

212: Fourth lens

213: Fourth mirror

214: First dichroic mirror

300: Objective lens

400: Two-photon microscopy unit

410: Second optical filter

420: Fifth lens

430: Second dichroic mirror

440: Third optical filter

450: First photomultiplier tube

460: Second photomultiplier tube

500: Reflectance confocal microscopy unit

510: Focus adjusting lens

520: Pin-hole

530: Third photomultiplier tube

600: Image generator

1. A combined reflectance confocal and two-photon microscopy system for non-invasive high-speed/high-contrast examination of living tissue, comprising:
a light source emitting laser beams;
an objective lens delivering light received from the light source to living tissue;
a two-photon microscopy unit photographing cells and extracellular matrix of the living tissue with effect light generated by two-photon fluorescence and SHG;
a reflectance confocal microscopy unit photographing an interior of the living tissue with effect light reflected from the living tissue;
an optical path guide guiding a traveling path of the laser beams from the light source to the objective lens and guiding the effect light generated from the living tissue to the two-photon microscopy unit and the reflectance confocal microscopy unit; and
an image generator generating an image of the living tissue photographed by the two-photon microscopy unit and the reflectance confocal microscopy unit.

2. The combined reflectance confocal and two-photon microscopy system according to claim 1, wherein the optical path guide comprises:
a first optical filter allowing only a laser beam having a wavelength of 680 nm or more among the laser beams emitted from the light source to pass therethrough;
a first lens group expanding the laser beams;
a beam splitter allowing the laser beams traveling from the light source to the objective lens to pass therethrough and guiding the effect light to the reflectance confocal microscopy unit;
a second lens group expanding the laser beams; and
a first dichroic mirror allowing the laser beams traveling from the light source to the objective lens to pass therethrough and guiding the effect light to the two-photon microscopy unit and the reflectance confocal microscopy unit.

3. A non-invasive high-speed/high-contrast examination method using a combined reflectance confocal and two-photon microscopy system for non-invasive high-speed/high-contrast examination of living tissue, the method comprising:
a living tissue staining step in which living tissue is stained with moxifloxacin;
an irradiation step in which a laser beam is emitted towards the living tissue;
an effect light guide step in which effect light reflected from the living tissue and subjected to two-photon excitation and SHG through moxifloxacin, intrinsic fluorophores, and intrinsic collagen is guided to a reflectance confocal microscopy unit and a two-photon microscopy unit;
an image generation step in which an image of the living tissue photographed by the two-photon microscopy unit and the reflectance confocal microscopy unit is generated,
wherein the effect light guide step comprises:
a first effect light guide step in which, among the effect light reflected from the living tissue and subjected to fluorescence excitation through moxifloxacin, first effect light having a wavelength of less than 700 nm is reflected from a first dichroic mirror and guided to the two-photon microscopy unit among the effect light reflected from the living tissue and subjected to fluorescence excitation through moxifloxacin; and
a second effect light guide step in which, among the effect light reflected from the living tissue, second effect light having a wavelength of 700 nm or more is allowed to pass through the first dichroic mirror and is guided to the reflectance confocal microscopy unit.

4. The non-invasive high-speed/high-contrast examination method according to claim 3, wherein the irradiation step comprises:
a first light filtering step in which, among the laser beams emitted from the light source, a laser beam having a wavelength of 680 nm or more is allowed to pass through a first optical filter;
a first beam expansion step in which the laser beam is primarily expanded by a first lens group;
a first beam passing step in which the laser beam subjected to primary expansion is allowed to pass through a beam splitter;
a second beam expansion step in which the laser beam is secondarily expanded by a second lens group;
a second beam passing step in which the laser beam subjected to secondary expansion is allowed to pass through a first dichroic mirror; and
a living tissue irradiation step in which the laser beam having passed through the first dichroic mirror is delivered to the living tissue through an objective lens.
